(19) United States
(12) Patent Application Publication
(10) Pub. No.: US 2025/0032762 A1
(43) Pub. Date: Jan. 30, 2025

(54) SYSTEMS AND METHODS FOR CHRONIC OBSTRUCTIVE PULMONARY DISEASE TARGETED LUNG DENERVATION

(71) Applicant: Medtronic Cryocath LP, Toronto (CA)

(72) Inventors: Joseph D. Brannan, Lyons, CO (US); Zhongping Yang, Woodbury, MN (US); Nicolas Coulombe, Anjou (CA); William J. Dickhans, Longmont, CO (US); Randal C. Schulhauser, Phoenix, AZ (US)

(21) Appl. No.: 18/843,355

(22) PCT Filed: Mar. 30, 2023
(86) PCT No.: PCT/CA2023/050427

§ 371 Date: Sep. 3, 2024

Related U.S. Application Data
(60) Provisional application No. 63/326,943, filed on Apr. 4, 2022.

(51) Int. Cl. A61M 25/10

(52) U.S. Cl. CPC A61M 25/10; A61M 2025/105; A61M 2025/1061; A61M 2210/1035

(57) ABSTRACT

A medical system includes a therapeutic fluid supply containing a therapeutic drug and a medical device in fluid communication with the therapeutic fluid supply. The medical device includes an expandable element and an injection tube coupled to the expandable element. The injection tube defines a port. The injection tube is in communication with the therapeutic fluid supply and configured to deliver the therapeutic drug through the port.

### CROSS-REFERENCE TO RELATED APPLICATION

The present application is related to and claims benefit under 35 U.S.C. § 119 (e) from U.S. Provisional Patent Application Ser. No. 63/326,943, filed 4 Apr. 2022, entitled “SYSTEMS AND METHODS FOR CHRONIC OBSTRUCTIVE PULMONARY DISEASE TARGETED LUNG DENERVATION”, the entire contents of which being incorporated herein by reference.

### FIELD

The present technology is generally related to devices, systems, and related methods thereof, for the denervation of lung tissue for the treatment of chronic obstructive pulmonary disease (COPD).

### BACKGROUND

COPD is a chronic inflammatory lung disease that causes obstructed airflow from the lungs. Symptoms include breathing difficulty, cough, mucus production and wheezing. People with COPD are at increased risk of developing heart disease, lung cancer, and other diseases. Targeted lung denervation (TLD) is a surgical procedure in which a catheter is advanced through a bronchoscope to a target area within a target bronchus to denervate the target area. Existing medical systems utilize thermal energy via cryogenic fluid and/or radiofrequency (RF) energy to denervate tissue. However, when applying thermal energy to a target treatment area, collateral tissues may be inadvertently affected.

### SUMMARY

The techniques of this disclosure generally relate to a system and device for targeted lung denervation, and methods of use thereof.

In one embodiment, a medical system includes a therapeutic fluid supply containing a therapeutic drug and a medical device in fluid communication with the therapeutic fluid supply. The medical device includes an expandable element and an injection tube coupled to the expandable element. The injection tube defines a port. The injection tube is in communication with the therapeutic fluid supply and configured to deliver the therapeutic drug through the port.

In one aspect of this embodiment, the medical device further includes an elongate body having a proximal portion, a distal portion opposite the proximal portion, and a lumen therethrough, and an inner shaft disposed within the lumen. The inner shaft has a distal portion proximate to the distal portion of the elongate body.

In another aspect of this embodiment, the medical system further includes an inflation fluid supply reservoir, and an inflation-deflation lumen extending through the inner shaft and out through the distal portion of the inner shaft. The inflation-deflation lumen defines a second port. The inflation-deflation lumen is in fluid communication with the inflation fluid reservoir and is configured to deliver inflation fluid through the second port to an inner chamber of the expandable element.

In another aspect of this embodiment, the expandable element surrounds at least a portion of the distal portion of the inner shaft.

In another aspect of this embodiment, the injection tube has a proximal portion disposed within the elongate body and a distal portion coupled to the expandable element.

In another aspect of this embodiment, an end portion of the injection tube extends outwardly from the expandable element.

In another aspect of this embodiment, the injection tube defines an injection lumen therethrough, and the end portion of the injection tube defines the port.

In another aspect of this embodiment, the therapeutic fluid supply is disposed within a fluid pump. The fluid pump is configured to initiate the delivery of the therapeutic drug through the injection tube.

In another aspect of this embodiment, the therapeutic drug is delivered through the injection tube at a pressure of approximately 50-2000 psi.

In another aspect of this embodiment, the injection tube tapers in diameter toward the end.

In another aspect of this embodiment, the medical device further includes a plurality of injection tubes spaced apart from one another around an outer surface of the expandable element.

In another aspect of this embodiment, each injection tube is composed of at least one of nitinol and a shape-memory alloy.

In yet another embodiment, a medical system for target lung denervation includes a therapeutic fluid supply containing a therapeutic drug and a medical device in fluid communication with the therapeutic fluid supply. The medical device includes an expandable element and an injection tube coupled to the expandable element. The injection tube defines a port. The injection tube is in communication with the therapeutic fluid supply and is configured to deliver the therapeutic drug through the port.

In one aspect of this embodiment, the medical system further includes a hub disposed between and in communication with the therapeutic fluid supply and the medical device.

In one aspect of this embodiment, the medical system further includes an inflation fluid supply reservoir in fluid communication with the medical device.

In another aspect of this embodiment, the hub includes a first portion coupled to the inflation fluid supply reservoir and a second portion coupled to the therapeutic fluid supply.

In another aspect of this embodiment, when inflated, the expandable element extends partially around an outer surface of the injection tube.

In another aspect of this embodiment, the injection tube has a proximal portion and a distal portion opposite the proximal portion. The distal portion of the injection tube is coupled to the expandable element.

In another aspect of this embodiment, the expandable element includes a recessed area. The distal portion of the injection tube being received within the recessed area.

In yet another embodiment, a method for targeted lung denervation includes providing a therapeutic fluid supply having a therapeutic drug and a medical device in fluid communication with the therapeutic fluid supply; advancing the medical device through a trachea of a mammal and positioning the expandable element within a target bronchus; inflating the expandable element so that the distal portion of the injection tube penetrates a wall of the target bronchus and is in contact with the area of target tissue; and delivering the therapeutic drug to the area of target tissue to denervate the target bronchus. The medical device includes an expandable element and an injection tube having a proximal portion and a distal portion. The distal portion is coupled to and extends outwardly from the expandable element and is configured to deliver the therapeutic drug to the area of target tissue within the target bronchus.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows an example medical system constructed in accordance with the principles of the present invention;

FIG. 2 shows a side view of medical device constructed in accordance with the principles of the present invention, the medical device having an expandable element and a plurality of injection tubes;

FIG. 3 shows a front section view A-A of the medical device of FIG. 1;

FIG. 4 shows the positioning of the medical device of FIGS. 1 and 3 within a target bronchus; and

FIG. 5 shows a flowchart of an example method constructed in accordance with the principles of the present invention.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

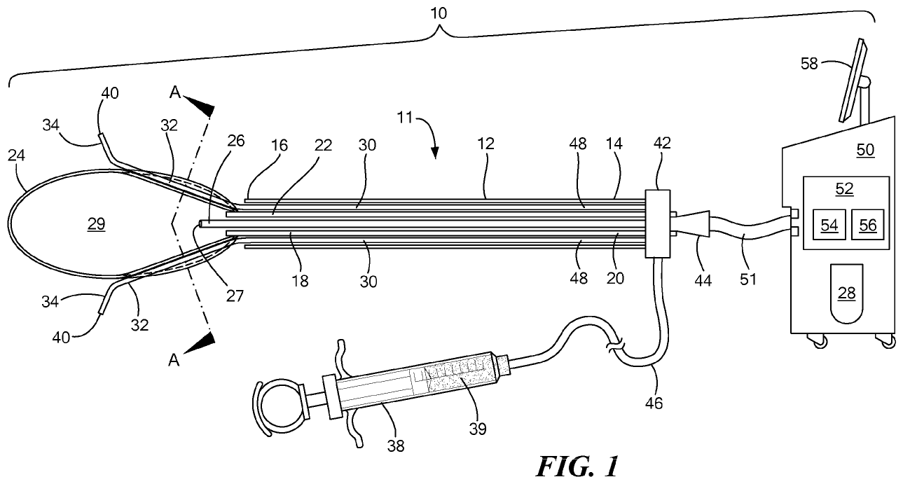

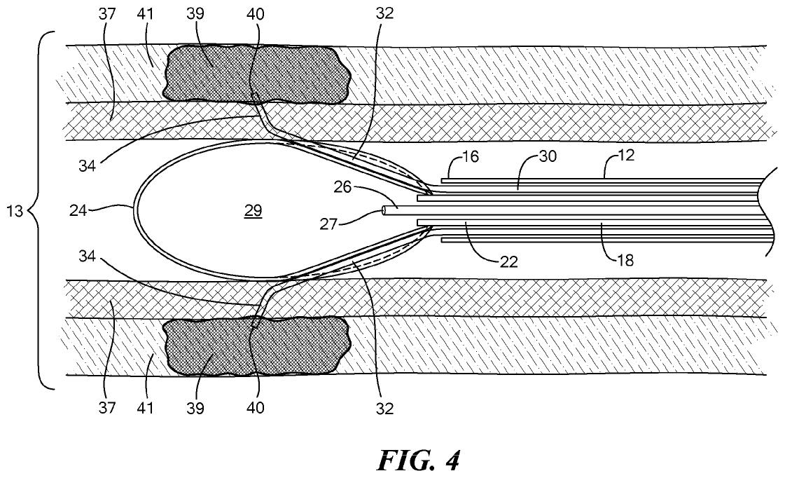

Referring now to the drawings in which like reference designators refer to like elements, there is shown in FIG. 1 an exemplary medical system for targeted lung denervation (TLD) constructed in accordance with the principles of the present application and designated generally as “10.” The medical system 10 is configured to be utilized inside the trachea and lungs of a mammalian patient for TLD. In one configuration, the medical system 10 includes a medical device 11 having an elongate body 12 sized and configured to be advanced through a trachea of a mammal and within a target bronchus 13 (shown in FIG. 4). The elongate body 12 includes a proximal portion 14 and a distal portion 16 opposite the proximal portion 14. The elongate body 12 defines a lumen sized and configured to allow for the passage of a rigid or semi-rigid inner shaft 18 that may be slidably and/or rotatably moveable with respect to the elongate body 12. The inner shaft 18 has a proximal portion 20 and a distal portion 22 proximate to the distal portion 16 of the elongate body 12. As shown in FIG. 1, the distal portion 22 of the inner shaft 18 at least partially extends through an opening defined by the distal portion 16 of the elongate body 12.

The system 10 further includes an expandable element 24, coupled to and/or partially surrounding the distal portion 22 of the inner shaft 18. The expandable element 24 may be a single or dual-walled balloon that is sized and configured to contact the wall of the target bronchus when inflated and/or create a seal within the target bronchus. The inner shaft 18 may further define a lumen sized and configured to allow for the passage of a guidewire and/or an inflation-deflation conduit or lumen 26 extending therein and out through the distal end of the distal portion 22 of the inner shaft 18. As shown in FIG. 1, in some configurations, the inflation-deflation lumen 26 may be configured for bidirectional flow, thus allowing for inflation and deflation of the expandable element 24 through the use of a single lumen. The inflation-deflation lumen 26 defines at least one opening, aperture, or port 27 that allows for inflation fluid to be delivered to, and exhausted from, an inner chamber 29 of the expandable element 24. The inflation-deflation lumen 26 may be directly coupled to an inflation fluid supply reservoir 28 or fluid source that contains fluid such as, for example, saline, air, or, refrigerant, or may be coupled to the inflation fluid supply reservoir 28 through the use of a hub as an intermediary (discussed in more detail below).

Continuing to refer to FIG. 1, the medical device 11 further includes at least one injection tube 30. Each injection tube 30 extends through the lumen of the elongate body 12 parallel to the inner shaft 18 and through the opening defined at the distal portion 16 of the elongate body 12. As shown in FIGS. 1-4, a distal portion 32 of each injection tube 30 is coupled, mounted, attached, or otherwise affixed to the outer surface of the expandable element 24. The distal portion 32 of each injection tube 30 includes an end portion 34 that is disposed at an angle and extends farther outward towards the target tissue (and away from the expandable element 24). In some configurations (for example, as shown in FIGS. 1-3) having more than one injection tube 30, each injection tube 30 may be spaced apart around the outer surface of the expandable element 24 such that no two injection tubes are in contact with one another. It is to be understood that although the injection tubes 30 may be uniformly spaced about the outer surface of the expandable element 24, the injection tubes 30 may also have uneven spacing between one another. In one example configuration, the medical device 11 may include four injection tubes 30. However, it is to be understood that the medical device 11 may also include 1, 2, 3, 5, 6, 7, 8, or any number of injection tubes 30 as desired by a clinician.

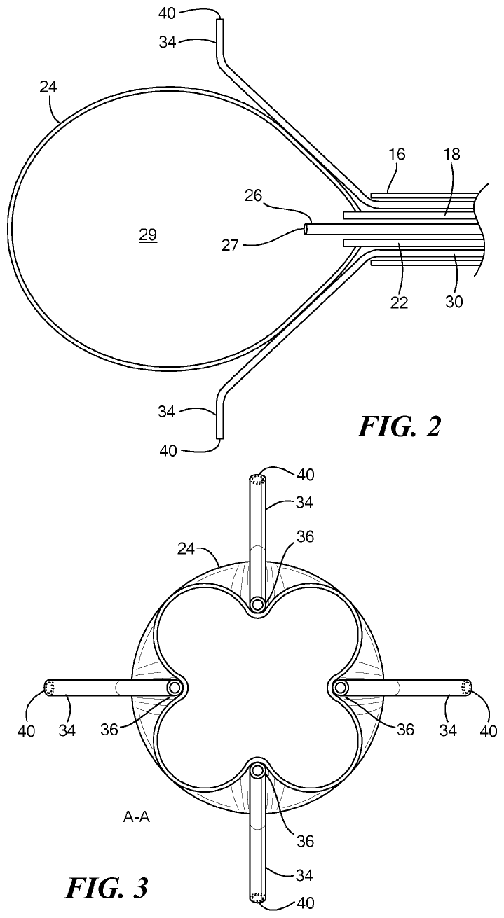

Now referring to FIGS. 1-3, as shown in FIG. 2, each injection tube 30 may be coupled to the expandable element 24 in such a manner that the injection tubes only marginally affect the outer diameter of the expandable element 24 when inflated. This may be the result of the injection tubes 30 being formed of a lightweight material that allows the expandable element 24 to readily inflate without experiencing enough resistance from the injection tubes 30 to indent or otherwise restrict the expansion of the expandable element 24. However, as shown in FIGS. 1 and 3, the injection tubes 30 may be configured such that when extended out of the distal portion 22 of the elongate body 12, the distal portions 32 of the injection tubes 30 may extend outwards until the tubes 30 reach a predetermined maximum diameter less than the maximum diameter of the expandable element 24. This causes the distal portions 32 to inwardly indent the expandable element 24 when the expandable element 24 is inflated (for example, as shown in FIG. 3). In other words, the injection tubes 30 may have a slight rigidity that causes the expandable element 24 to partially surround each injection tube 30 when the expandable element 24 is inflated. As a result, in some embodiments, when inflated the expandable element 24 may define a recessed portion 36 or groove that is sized and configured to receive the distal portion 32 of a respective injection tube 30 when the injection tube 30 is coupled to the expandable element 24. The injection tube 30 may remain within the recession 36 or groove throughout the entire length of the treatment procedure (i.e., during the inflation phase and treatment phase) and be partially surrounded by at least a portion of the expandable element 24. As shown in FIG. 4, the end portion 34 of each injection tube 30 extends outwardly towards the bronchial wall 37 such that, as the expandable element 24 is inflated, the end portion 34 comes into contact with and penetrate the smooth muscle tissue of the bronchial wall 37 so that therapeutic fluid 39 can be delivered to the target tissue 41, which in some embodiments includes the cartilage. However, in other configurations, the end portion 34 may instead contact, but not penetrate, the bronchial wall 37, or the end portion 34 may be in close proximity to but not penetrate the bronchial wall 37. Additionally, in some configurations, the distal portion 32 of each injection tube 30 may taper inwards towards the end portion 34 to define a sharpened or pointed edge to more easily penetrate the bronchial wall 37. However, in other configurations, the injections tubes 30 may have a uniform diameter through their entirety such that the distal portions 32 do not form a sharpened or pointed edge at the end portion 34. It is to be understood that although the bronchial wall 37 and target tissue 41 have been shown as shaded regions for ease of illustration, the bronchial wall 37 and target tissue 41 may each have unique shapes, sizes, and curvatures that are unique to each particular patient.

Each injection tube 30 may be composed of at least one of nitinol and a shape-memory alloy so that the tube 30 may be advanced and retracted within the elongate body 12 before and after the treatment procedure. When advancing the injection tube through the elongate body 12, the distal portions 32 are substantially linear. However, because the end portions 34 are biased to extend outwards to contact tissue, they transition from the substantially linear configuration to the extended or protruding configuration (as shown in FIG. 1) once the end portion 34 of each injection tube 30 passes through the opening defined at the distal portion 14 of the elongate body 12. Each injection tube 30 defines an injection lumen in fluid communication with a therapeutic fluid supply 38 (e.g., syringe, fluid chamber, fluid reservoir, fluid pump) containing therapeutic fluid 39 and an aperture, opening or port 40 at the end portion 34 sized and configured to allow the therapeutic fluid 39 to be delivered through the injection lumen 30 and to the area of target tissue 41. As described herein, the therapeutic fluid 39 contains a therapeutic drug or chemical (e.g., alcohol, acetic acid, phenol, glycerol, hypertonic saline, and Botox®) capable of denervating target tissue. Additionally, in other embodiments, the therapeutic fluid 39 may be a chemical adjuvant capable of rendering treated tissue more susceptible to ablation therapies and may inhibit the rate at which tissue regrowth occurs at the ablation site.

Referring again to FIG. 1, the system 10 further includes a hub 42 disposed between the inflation fluid supply reservoir 28, the therapeutic fluid supply 38 and the elongate body 12. The hub 42 provides for an inflow-outflow connector 44 that is coupled to the inflation fluid reservoir and allows the inflation-deflation lumen 26 to be in fluid communication with the inflation fluid supply reservoir 28. The hub 42 also includes an opening or aperture sized and configured to receive a portion of a delivery conduit 46 coupled to the therapeutic fluid supply 38. The proximal portion 14 of the elongate body 12 is coupled to the hub 42 and the hub 42 is sized and configured to receive the proximal portion 20 of the inner shaft 18 and a proximal portion 48 of each injection tube 30.

In some embodiments, the inflation fluid supply reservoir 28 may be disposed within a console 50. The console 50 may be coupled or connected to the inflow-outflow connector 44 of the hub 42 by one or more cables, conduits, or connectors 51. The console 50 may include processing circuitry 52 having processor 54 and a memory 56 in communication with the processor 54. The memory 56 may be programmed and/or configured to store instructions that, when executed by the processor 54, configure the processor 54 to execute instructions or algorithms to provide for the automated operation and performance of the features, sequences, calculations, or procedures described herein and/or required for a given medical procedure, such as, for example, initiating the inflation and/or deflation of the expandable element 24. As shown in FIG. 1, the console 50 may further include or be in electrical communication with one or more other system components, such as one or more displays 58, user input devices, a mapping and/or navigation system for collecting and conveying information from and to the user. As used herein, the term “console 50” for simplicity may include any system components that are not part of the medical device 11 itself, other than the therapeutic fluid supply 38, regardless of whether the component is physically located within or external to the console 50. In embodiments that include a navigation system (not shown), the navigation system may be a standalone system in communication with the console 50 or may be contained within or integrated with the console 50. The console 50 may also include one or more components for the delivery of one or more energy modalities for which the system is used. For example, if the system 10 is also used to deliver cryotherapy, the console 50 may include a supply of cryogenic fluid such as a coolant, cryogenic refrigerant, or the like, an exhaust or scavenging system for recovering or venting expended fluid for re-use or disposal, as well as various control mechanisms. However, in some embodiments, the inflation fluid supply reservoir 28 may the supply of cryogenic fluid that is used to both inflate the expandable element 24 and ablate or denervate target tissue. In addition to providing an exhaust function for the fluid or coolant supply, the console 50 may also include pumps, valves, controllers or the like to recover and/or recirculate fluid delivered to the fluid pathways of the medical device 11. Further, a vacuum pump (not shown) in the console 50 may create a low-pressure environment in one or more conduits within the medical device 11 so that fluid is drawn into the conduit(s)/lumen(s) of the elongate body 12, away from the distal portion 16 and towards the proximal portion 14 of the elongate body 12. Additionally, or alternatively, the console 50 may include an energy source as a treatment or diagnostic mechanism in communication with a treatment electrode (not shown) disposed around an outer surface of the expandable element 24. Also, the energy source (not shown) may be a radiofrequency generator having a plurality of output channels, with each channel coupled to the individual treatment electrode. The radiofrequency generator may be operable in one or more modes of operation.

In some embodiments, the therapeutic fluid supply 38 may be a supply of therapeutic fluid disposed within a high-pressure pump (e.g., high-pressure syringe pump) configured to deliver the therapeutic fluid through the injection tubes 30 in a high pressure jet (delivered at, for example, 50-2000 psi) to form a pin hole in and penetrate beyond the tissue/vessel wall of the area of target tissue. In some such embodiments, the end portion 34 of each injection tube 30 may be in close proximity to, but not in contact with, the area of target tissue. This may help reduce or eliminate the risk associated with penetrating tissue within the injection tube 30. For example, because the injection tube 30 does not penetrate tissue when delivering the high pressure jet, there is less risk when concluding the treatment procedure because the injection tube 30 does not need to be removed from penetrated tissue (and possibly damaging collateral tissue upon removal). Additionally, delivery of the therapeutic drug in a high pressure jet may result in enhanced distribution of the drug when compared to delivery administered via tissue penetration.

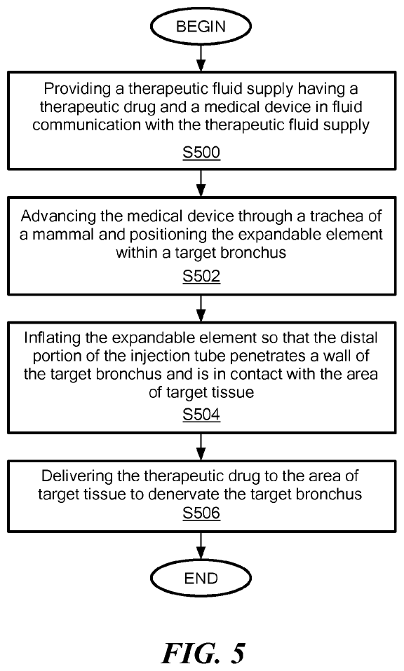

Referring now to FIG. 5, an example method according to the principles of the present application is shown. The method includes providing a medical device 11 in communication with a console 50 having the inflation fluid reservoir 28 disposed therein and a therapeutic fluid supply 38 (Block S500). The medical device 11, including the expandable element 24 and injection tubes 30, is then advanced through the trachea of a patient and positioned proximate to an area of target tissue within a target bronchus (Block S502). Once in the desired position, the processing circuitry 52 of the console 50 is configured to initiate the delivery of inflation fluid to the medical device 11 to inflate the expandable element 24 such that the end portion 34 of each injection tube 30 penetrates the tissue wall of the area of target tissue (Block S504). A clinician may then manually or automatically initiate the delivery of fluid from the therapeutic fluid supply 38 to the medical device 11 and through the injection tubes 30 until the therapeutic fluid is dispersed within the area of target tissue and denervates the target bronchus (Block S506).

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A medical system, comprising:
a therapeutic fluid supply containing a therapeutic drug;
a medical device in fluid communication with the therapeutic fluid supply and including:
an expandable element; and
an injection tube coupled to the expandable element and defining a port, the injection tube being in fluid communication with the therapeutic fluid supply and configured to deliver the therapeutic drug through the port.

2. The medical system of claim 1, wherein the medical device further includes:
an elongate body having a proximal portion, a distal portion opposite the proximal portion, and a lumen therethrough; and
an inner shaft disposed within the lumen, the inner shaft having a distal portion proximate to the distal portion of the elongate body.

3. The medical system of claim 2, further including:
an inflation fluid supply reservoir; and
an inflation-deflation lumen extending through the inner shaft and out through the distal portion of the inner shaft, the inflation-deflation lumen defining a second port, the inflation-deflation lumen being in fluid communication with the inflation fluid reservoir and configured to deliver inflation fluid through the second port to an inner chamber of the expandable element.

4. The medical system of claim 3, wherein the expandable element surrounds at least a portion of the distal portion of the inner shaft.

5. The medical system of claim 4, wherein the injection tube has a proximal portion disposed within the elongate body and a distal portion coupled to the expandable element.

6. The medical system of claim 5, wherein an end portion of the injection tube extends outwardly from the expandable element.

7. The medical system of claim 6, wherein:
the injection tube defines an injection lumen therethrough; and
the end portion of the injection tube defines the port.

8. The medical system of claim 7, wherein the therapeutic fluid supply is disposed within a fluid pump, the fluid pump being configured to initiate the delivery of the therapeutic drug through the injection tube.

9. The medical system of claim 8, wherein the therapeutic drug is delivered through the injection tube at approximately 50˜2000 psi.

10. The medical system of claim 9, wherein the injection tube tapers in diameter toward the end portion.

11. The medical system of claim 10, wherein the medical device further includes a plurality of injection tubes spaced apart from one another around an outer surface of the expandable element.

12. The medical system of claim 11, wherein each injection tube is composed of at least one of nitinol and a shape-memory alloy.

13. A medical system for targeted lung denervation, comprising:
a therapeutic fluid supply containing a therapeutic drug; and
a medical device in fluid communication with the therapeutic fluid supply and including:
an expandable element; and
an injection tube coupled to the expandable element and defining a port, the injection tube being in communication with the therapeutic fluid supply and configured to deliver the therapeutic drug through the port.

14. The medical system of claim 13, further including a hub disposed between and in communication with the therapeutic fluid supply and the medical device.

15. The medical system of claim 14, further including an inflation fluid supply reservoir in fluid communication with the medical device.

16. The medical system of claim 15, wherein the hub includes a first portion coupled to the inflation fluid supply reservoir and a second portion coupled to the therapeutic fluid supply.

17. The medical system of claim 16, wherein, when inflated, the expandable element extends partially around an outer surface of the injection tube.

18. The medical system of claim 16, wherein the injection tube has a proximal portion and a distal portion opposite the proximal portion, the distal portion of the injection tube being coupled to the expandable element.

19. The medical system of claim 18, wherein the expandable element includes a recessed area, the distal portion of the injection tube being received within the recessed area.

20. A method for targeted lung denervation, comprising:
providing a therapeutic fluid supply having a therapeutic drug and a medical device in fluid communication with the therapeutic fluid supply, the medical device including:
an expandable element; and
an injection tube having a proximal portion and a distal portion, the distal portion being coupled to and extending outwardly from the expandable element and defining a port configured to deliver the therapeutic drug to an area of target tissue within a target bronchus;
advancing the medical device through a trachea of a mammal and positioning the expandable element within the target bronchus;
inflating the expandable element so that the distal portion of the injection tube penetrates a wall of the target bronchus and is in contact with the area of target tissue; and
delivering the therapeutic drug to the area of target tissue to denervate the target bronchus.
